(19) United States
(12) Patent Application Publication
(10) Pub. No.: US 2006/0095079 A1
(43) Pub. Date: May 4, 2006

(54) Sub-mucosal medical device implantation

(76) Inventor: Martin T. Gerber, Maple Grove, MN (US)

Correspondence Address: SHUMAKER & SIEFFERT, P. A., 8425 SEASONS PARKWAY, SUITE 105, ST. PAUL, MN, 55125, US

(21) Appl. No.: 10/977,501

(22) Filed: Oct. 29, 2004

(51) Int. Cl. A61N 1/00

(52) U.S. Cl. 607/2

(57) ABSTRACT

In general, the invention is directed to devices and methods for use in sub-mucosal implantation of medical devices such as electrical stimulators and physiological sensors. An implantation instrument includes a probe defining a cavity, and a vacuum channel to apply vacuum pressure to draw tissue into the cavity. Once the tissue is captured within the cavity, a needle is advanced through the probe to define an implantation pocket. Then, a medical device, such as an electrical stimulator or sensor, is advanced through the probe and into the pocket. In this manner, a medical device can be quickly and securely implanted at a desired tissue site within a patient.

### TECHNICAL FIELD

The invention relates generally to implantable medical devices, and, more particularly, to techniques for implantation of a medical device.

### BACKGROUND

Implantable medical devices, such as sensors, neurostimulators, and drug delivery pumps, are implanted within patients to alleviate a variety of disorders. Examples of disorders treatable with implantable medical devices include pelvic floor disorders, such as urinary or fecal incontinence, interstitial cystitis, sexual dysfunction, and pelvic pain, neurological disorders such as chronic pain, epilepsy and Parkinson's disease, gastrointestinal disorders such as gastroparesis, and cardiac disorders such as bradycardia, tachycardia and arrhythmia.

Electrical stimulation of various nerve sites can offer relief for different disorders. For example, spinal cord stimulation can alleviate chronic pain, while stimulation of the sacral nerves, pudendal nerves, and other nerves of the pelvic floor has been found to offer relief for many pelvic floor disorders. An example of an existing neurostimulation system for spinal cord stimulation is the Medtronic Synergy therapy system marketed by Medtronic, Inc. of Minneapolis, Minn. An example of an existing neurostimulation system for treatment of urinary urge incontinence is the Medtronic Interstim therapy system marketed by Medtronic, Inc.

Neurostimulation systems with multiple, small, self-contained neurostimulators also have been proposed. For example, U.S. Pat. No. 6,185,452 to Schulman et al. describes implantation of one or more miniature stimulators, referred to as microstimulators, with external electrodes for nerve or muscle stimulation. U.S. Pat. No. 6,650,943 to Whitehurst et al. describes implantation of microstimulators to treat erectile dysfunction. U.S. Pat. No. 6,735,474 to Loeb et al. describes a microstimulator system for treatment of urinary incontinence. Microstimulators can be implanted surgically or by injection into a desired tissue site.

Implantation of small sensors to sense physiological signals for wireless transmission to microstimulators or external receivers has also been proposed. For example, U.S. Pat. No. 6,650,943 to Whitehurst et al. describes implantation of sensors that communicate with a stimulator. U.S. Pat. No. 6,689,056 to Kilcoyne et al. describes fixation of a sensor within the esophagus for transmission of measurements to an external receiver.

Table 1 below lists documents that disclose various techniques for implantation of neurostimulators and sensors.

TABLE 1

| Patent | | |
|---|---|---|
| Number | Inventors | Title |
| 6,185,452 | Schulman | Battery-powered patient implantable device |
| | et al. | |
| 6,650,943 | Whitehurst | Fully implantable neurostimulator for |
| | et al. | cavernous nerve stimulation as a therapy for |
| | | erectile dysfunction and other sexual |
| | | dysfunction |
| 6,735,474 | Loeb et al. | Implantable stimulator system and method for |
| | | treatment of incontinence and pain |
| 6,689,056 | Kilcoyne | Implantable monitoring probe |
| | et al. | |

All documents listed in Table 1 above are hereby incorporated by reference herein in their respective entireties. As those of ordinary skill in the art will appreciate readily upon reading the Summary, Detailed Description and Claims set forth below, many of the devices and methods disclosed in the patents of Table 1 may be modified advantageously by using the techniques of the present invention.

### SUMMARY

The invention is directed to an apparatus and method for implantation of implantable medical devices such as electrical stimulators and physiological sensors.

Various embodiments of the present invention provide solutions to one or more problems existing in the prior art with respect to implantation of medical devices. The problems include, for example, pain, discomfort, potential complications and recovery time associated with surgical implantation techniques. Additional problems include difficulty in stabilizing a target site for surgical implantation or implantation by injection, and inadequate precision in the location and depth of the resulting implantation. These problems, in turn, can result in improper or insecure placement of an implantable medical device within the patient, undermining the efficacy or longevity of the implanted medical device.

Various embodiments of the present invention are capable of solving at least one of the foregoing problems. In general, the invention is directed to a device and method for use in sub-mucosal implantation of medical devices such as electrical stimulators and physiological sensors. An implantation instrument includes a probe defining a cavity, and a vacuum channel to apply vacuum pressure to draw tissue into the cavity. Once the tissue is captured within the cavity, a hollow stylet may be advanced through the probe to inject fluid and expand the mucosal and submucosal tissue to form an implantation pocket. Then, an electrical stimulator or sensor is advanced through the probe and into the pocket. In this manner, a stimulator or sensor can be quickly and securely implanted at a desired tissue site within a patient.

In one embodiment, the invention provides a method for implanting a medical device, the method comprising applying vacuum pressure to a mucosal layer to draw a portion of the mucosal tissue layer and a portion of a sub-mucosal layer into a vacuum cavity defined by an implantation instrument, and introducing a medical device into the portion of the sub-mucosal tissue layer drawn into the vacuum cavity, wherein the medical device includes an electrically powered medical device.

In another embodiment, the invention provides a system for implantation of a medical device, the system comprising an elongated delivery tube, a probe coupled to a distal end of the delivery tube, a vacuum cavity defined by the probe to apply vacuum pressure to a mucosal layer to draw a portion of the mucosal tissue layer and a portion of a sub-mucosal layer into a vacuum cavity defined by an implantation instrument, a vacuum channel to apply the vacuum pressure to the vacuum cavity, an electrically powered medical device, and a working channel to introduce the medical device into the portion of the sub-mucosal tissue layer drawn into the vacuum cavity.

In comparison to known techniques for implantation of medical devices, such as stimulators or sensors, various embodiments of the invention may provide one or more advantages. For example, the application of vacuum pressure may be used to stabilize a tissue site for more secure and precise implantation of the medical device. In addition, the vacuum cavity and associated structure of the probe can be selectively sized to permit more accurate implantation of a medical device at a precise depth within a sub-mucosal tissue site. More secure, precise placement can enhance the efficacy and reliability of the implanted medical device for the patient. Also, sub-mucosal implantation may permit implantation of medical devices within a body lumen, such as the urethra, colon, trachea, or esophagus, with reduced obstruction of the lumen. Further, the implantation instrument facilitates the quick and simple placement of not only a single medical device, but also a series of medical devices, such as stimulators and sensors, to be implanted within a region of the body.

The above summary is not intended to describe each embodiment or every embodiment of the present invention or each and every feature of the invention. Advantages and attainments, together with a more complete understanding of the invention, will become apparent and appreciated by referring to the following detailed description and claims taken in conjunction with the accompanying drawings.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram illustrating sub-mucosal implantation of a medical device.

FIG. 2 is a schematic diagram illustrating a probe for sub-mucosal implantation of the medical device of FIG. 1.

FIG. 3 is a schematic diagram of an implantation instrument incorporating the probe of FIG. 2.

FIG. 4 is a bottom plan view of a vacuum cavity in the probe of FIG. 2.

FIG. 5 is a cross-sectional view taken along line A-A′ of FIG. 4.

FIG. 6 is a cross-sectional diagram illustrating use of the probe of FIG. 2 to form a sub-mucosal implantation pocket.

FIG. 7 is a cross-sectional diagram illustrating introduction of a medical device into the sub-mucosal implantation pocket of FIG. 6.

FIG. 8 is a cross-sectional diagram illustrating placement of a medical device within the sub-mucosal implantation pocket of FIG. 6.

FIG. 9 is a diagram illustrating sub-mucosal implantation of medical devices within the urinary tract.

FIG. 10 is a diagram illustrating an implantation instrument for implanting medical devices within the bladder and urethra.

FIG. 11 is a schematic diagram illustrating an implantable stimulator.

FIG. 12 is a schematic diagram illustrating an implantable sensing device.

FIG. 13 is a schematic diagram illustrating an implantable module incorporating a stimulator and a sensor.

FIG. 14 is a block diagram illustrating a stimulator for sub-mucosal implantation within a patient.

FIG. 15 is a block diagram illustrating a sensing device for sub-mucosal implantation within a patient.

FIG. 16 is a block diagram illustrating an array of sub-mucosally implanted stimulators and sensors.

FIG. 17 is a flow diagram illustrating a technique for sub-mucosal implantation of medical devices.

### DETAILED DESCRIPTION

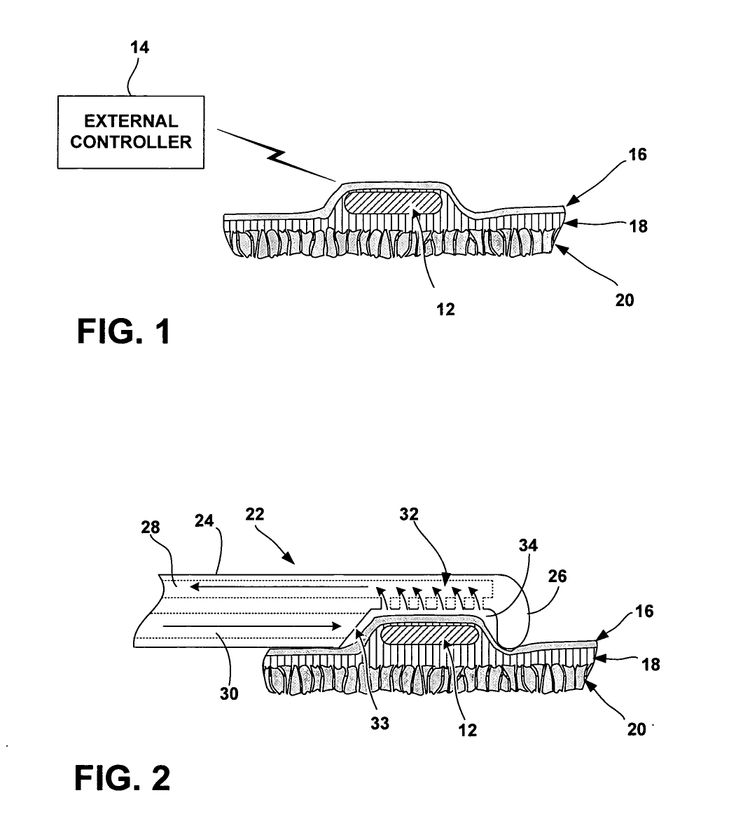

FIG. 1 is a schematic diagram illustrating sub-mucosal implantation of a medical device 12. As shown in FIG. 1, medical device 12 is beneath a mucosal layer 16 and within a sub-mucosal layer 18 that resides above a muscle layer 20. Mucosal layer 16 may be located on the interior or exterior of any of a variety of body lumens such as the esophagus, stomach, intestines, urethra, bladder, colon, or nasal passages, as well as on the interior or exterior of a variety of body organs such as the liver, pancreas, kidneys, or heart. Sub-mucosal implantation permits secure fixation of medical device 12 for delivery of therapy or sensing of physiological conditions. For example, medical device 12 may take the form of an electrical stimulator, a physiological sensor, a drug delivery device, or a combination of two or more of such devices. However, application of the invention for electrical stimulators and sensors will be described for purposes of illustration.

As shown in FIG. 1, medical device 12 may have a capsule-like housing, although the shape and size of the medical device may vary. For example, the housing may be disc-shaped or spherically shaped. Also, in some embodiments, the housing may include one or more surface irregularities, such as protrusions, to help anchor the housing within sub-mucosal tissue. Medical device 12 includes a telemetry interface for communication with an external device or other implanted devices. When constructed as a stimulator, for example, medical device 12 may receive commands from an external controller 14 by wireless telemetry, and also communicate operational information to the external controller. In some embodiments, external controller 14 may control medical device 12 in real-time, or download programs or parameters to be applied by the medical device during delivery of electrical stimulation therapy.

Medical device 12 may be configured, for example, to deliver neurostimulation to selected nerve sites or muscle stimulation to selected muscles. Medical device 12 may deliver stimulation to provide therapy for a variety of disorders including pelvic floor disorders, such as urinary or fecal incontinence, interstitial cystitis, sexual dysfunction, and pelvic pain, neurological disorders such as chronic pain, epilepsy and Parkinson's disease, gastrointestinal disorders such as gastroparesis, and cardiac disorders such as bradycardia, tachycardia and arrhythmia. Accordingly, medical device 12 may be implanted in a variety of different positions throughout a patient's body

Alternatively, medical device 12 may be constructed as a sensor to sense physiological conditions in the area near the implantation site. For example, medical device 12 may measure pressure, flow, force, volume, electromyographic potentials, pH, impedance, fluid level, fluid presence, or the like. Medical device 12 may communicate physiological information by wireless telemetry to an external receiver for additional processing. In some embodiments, multiple sensors may be implanted at different sub-mucosal implantation sites to sense physiological conditions in different areas. Alternatively, multiple sensors with different types of sensors may be implanted sub-mucosally to sense different types of physiological conditions in a common area.

In other embodiments, medical device 12 may integrate both a stimulator and sensor, or multiple stimulators or sensors. In each case, medical device 12 may be self-powered, e.g., by a rechargeable or non-rechargeable battery. The battery may be recharged telemetrically by an inductive power interface, which may be desirable for applications in which medical device is intended to function over an extended period of time. For example, an external device may include an inductive coil that is placed in proximity to medical device 12 for inductive transfer of power. Alternatively, medical device 12 may not have a battery, and instead may be powered telemetrically by an inductive power interface. As a further alternative, medical device 12 may be designed to deliver a therapeutic substance into sub-mucosal layer 18, e.g., by pumping or eluting the substance into the sub-mucosal tissue.

FIG. 2 is a schematic diagram illustrating a probe 22 for sub-mucosal implantation of medical device 12 of FIG. 1. As shown in FIG. 2, probe 22 includes a probe housing 24 with a distal tip 26. Probe housing 24 defines a vacuum channel 28 and a working channel 30. A plurality of suction ports 32 extend into a vacuum cavity 34, and are in fluid communication with vacuum channel 28. Vacuum cavity 34 serves as a tissue securing mechanism to capture and stabilize a portion of the mucosal tissue layer 16 and sub-mucosal tissue layer 18 for preparation of an implantation site. Working channel 30 defines an opening 33 for insertion of implantation tools and, ultimately, medical device 12, into sub-mucosal layer 18.

Probe 22 may be sized for introduction via a variety of different body lumens, such as the esophagus, stomach, intestines, urethra, bladder, colon, or nasal passages. Alternatively, in some embodiments, probe 22 may be introduced via surgical openings or laparoscopic ports. In either case, vacuum cavity 34 can be sized to achieve a desired implantation depth. For example, vacuum cavity 34 may be constructed with a depth and volume designed to capture a precise amount of tissue. In this manner, the position of working channel 30 and opening 33 relative to the captured tissue will generally determine the depth at which medical device 12 is implanted within sub-mucosal layer 18.

In operation, a proximal end of vacuum channel 28 is coupled to a source of vacuum pressure. In this manner, vacuum channel 28 applies the vacuum pressure to mucosal tissue 16 via vacuum ports 32, thereby drawing the mucosal layer, and sub-mucosal layer 18 into vacuum cavity 34. Once mucosal layer 16 and sub-mucosal layer 18 are captured within vacuum cavity 34, implantation tools are introduced via working channel 30 to prepare an implantation pocket for medical device 12 and place the medical device within the implantation pocket. Hence, probe 22 stabilizes the capture tissue while medical device 12 is implanted. Following implantation of medical device 12, probe 22 can be withdrawn, leaving the medical device in place for stimulation, drug delivery or sensing applications, as applicable.

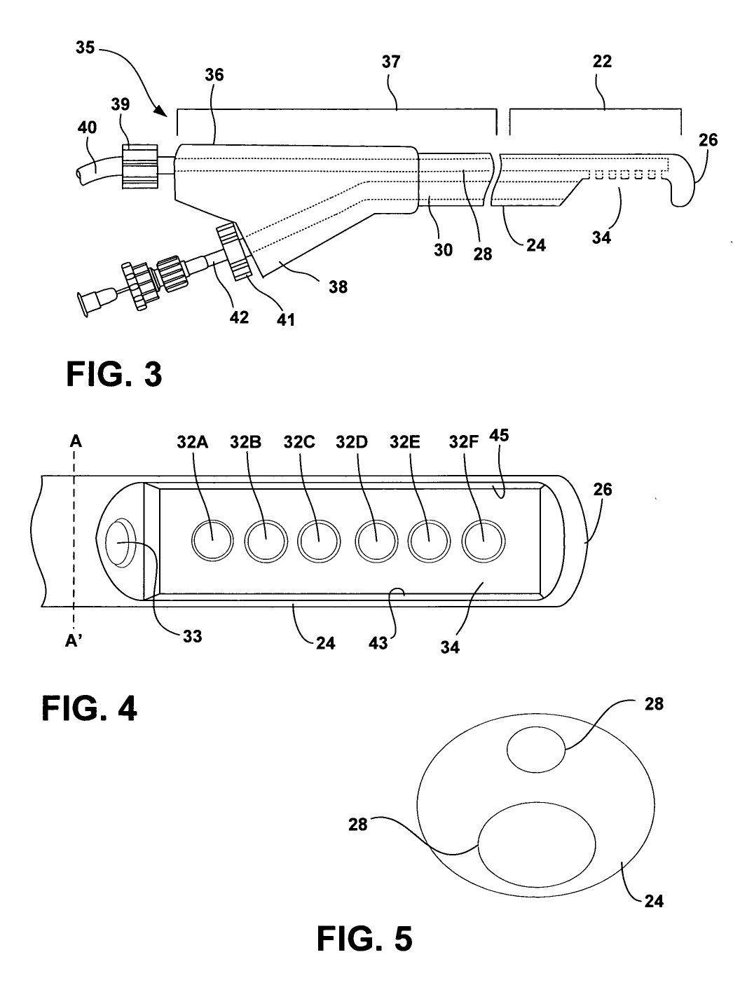

FIG. 3 is a schematic diagram of an implantation instrument 35 incorporating probe 22 of FIG. 2. As shown in FIG. 3, probe 22 is positioned at the distal end of an elongated delivery tube 37. A proximal end of delivery tube 37 is coupled to a handle 36. A user manipulates handle 36 to guide distal probe 22 to a target tissue site for implantation of medical device 12. Handle 36 includes a fluid coupling member 39 that receives an external vacuum channel 40 for fluid connection to vacuum channel 38 within delivery tube 37. A mechanical coupling member 41 is provided to permit insertion of different working tools into working channel 30 of delivery tube 37.

Tube 37 and probe 22 may be formed from any of a variety of different biocompatible materials. In general, tube 37 and probe 22 may be substantially flexible, but should have sufficient structural integrity to withstand vacuum pressure applied via vacuum channel 28 and vacuum ports 32. Example materials for fabrication of probe 22 and tube 37 include polyurethrane and silicone. In some embodiments, probe 22 may be more less compliant that tube 37, and may be formed of include biocompatible metals or polymers that are resilient to deformation under vacuum pressure, such as such as polycarbonate, acrylic, high-density polyethylene (HDPE), nylon, polytetrafluorethylene (PTFE), stainless steel, or titanium. Probe 22 may be molded and attached to tube 37, which may be extruded.

Implantation instrument 35 may include endoscopic visualization features that permit the user to view the target tissue site. For example, an endoscopic viewing scope may be deployed via working channel 30 to view the progress of probe 22 relative to the target site, and observe the tissue drawn into vacuum cavity 34. Once the user has verified that the tissue is properly captured within vacuum cavity 34, the rest of the implantation procedure may proceed blindly without further visualization, although the scope can be reintroduced, desired, to verify proper implantation or proper preparation of the implantation pocket.

Delivery tube 37 and probe 22 are sized according to the intended implantation procedure. For example, delivery tube 37 and probe 22 may be sized for introduction into the esophagus, either through the nasal or oral passage, the colon, or the male or female urethra. For urethral introduction, the diameter of delivery tube 37 and probe 22 may be very small. For surgical or laparoscopic introduction, however, the diameter of delivery tube 37 and probe 22 may be substantially larger. Similarly, the length of delivery tube 37 will vary according to the implantation site, and may range from a 5 to 20 centimeters for urethral, nasal or colon applications to 100 to 300 centimeters for esophageal, stomach, or intestinal applications. Delivery tube 37 may be formed from a flexible material to reduce trauma or discomfort for the patient, and permit negotiation of curves within a body lumen of the patient.

FIG. 4 is a bottom plan view of a vacuum cavity 34 in probe 22 of FIG. 2. As shown in FIG. 4, vacuum cavity 34 is defined by a pair of side walls 43, 45. FIG. 5 is a cross-sectional view of probe 22 taken along line A-A′ of FIG. 4, showing vacuum channel 28 and working channel 30. Vacuum ports 32A-32E open into vacuum cavity 34 to apply suction to mucosal and sub-mucosal tissue. Working channel 30 has an opening 33 that opens at a proximal end of vacuum cavity 34. In the example of FIG. 4, vacuum cavity 34 includes six vacuum ports 32. However, the particular number and arrangement of vacuum ports 32 may be subject to variation, and should not be considered limiting of the invention. In addition, although vacuum cavity 34 is shown as being substantially rectangular, other shapes may be used for the vacuum cavity.

As an example, vacuum cavity 34 may have an length along the longitudinal axis of probe 22 of approximately 5 to 40 mm, a width in a direction perpendicular to the length of approximately 5 to 15 mm, and a depth from a point at which walls 43, 45 contact tissue to vacuum ports 32 of approximately 5 to 15 mm. As a further example, probe 22 may have an average diameter of approximately 10 to 25 mm to permit passage through a body lumen. These dimensions permit probe 22 to capture a sufficient volume of tissue to obtain access to sub-mucosal layer 18 for implantation of medical device 12. However, such dimensions may be subject to significant variation in light of the particular applications and the unique characteristics that may be presented by different implantation sites within the body of the patient. For example, implantation within the urethra may present more aggressive size limitations that implantation via the esophagus.

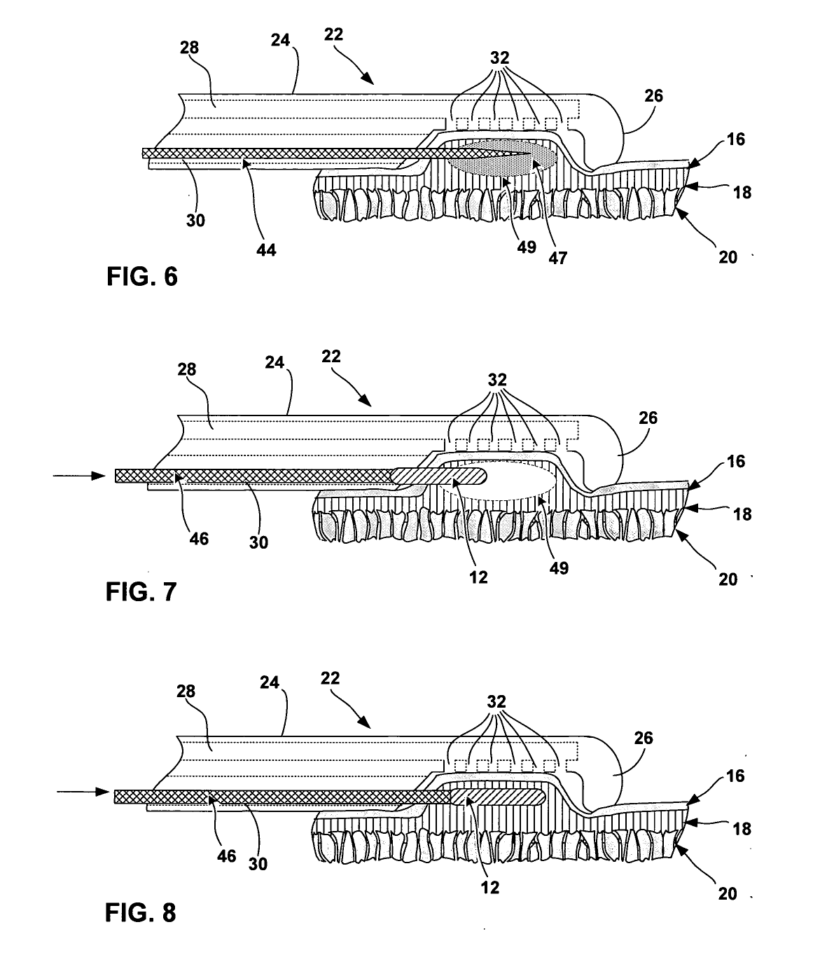

FIG. 6 is a cross-sectional diagram illustrating use of probe 22 of FIG. 2 to form a sub-mucosal implantation pocket. In the example of FIG. 6, vacuum pressure has been applied via vacuum channel 28 and vacuum ports 32 to draw a section of mucosal layer 16 and sub-mucosal layer 18 into vacuum cavity 34. In this manner, probe 22 draws a controlled amount of tissue into vacuum cavity 34 and stabilizes the tissue for the implantation procedure. Accordingly, medical device 12 can be implanted in a simple and convenient procedure that also offers significant precision in terms of the depth at which the medical device is implanted.

To form an implantation pocket 47, a stylet 44 in introduced into working channel 30 and inserted into the captured tissue. Then, a fluid such as saline is introduced into the capture tissue via stylet 44 to expand the tissue and form an implantation pocket 49. The introduction of a fluid to form the implantation pocket 49 may be optional. In particular, for some implantation sites, the formation of an implantation pocket 49 may be unnecessary. Instead, the captured tissue may be resected with a cutting tool introduced via working channel 30, thereby creating an opening for implantation of medical device 12, particularly when the medical device is small. As a further alternative, in some embodiments, it may be unnecessary to form an implantation pocket or resect the captured tissue. Instead, medical device 12 may be shaped such to facilitate penetration of the tissue. For example, one end of medical device 12 may be sharpened or pointed to facilitate penetration.

FIG. 7 is a cross-sectional diagram illustrating introduction of medical device 12 into the sub-mucosal implantation pocket 49 of FIG. 6. A push rod 46 is advanced along the length of working channel 30 to move medical device 12 into implantation pocket. FIG. 8 is a cross-sectional diagram illustrating placement of medical device 12 within the sub-mucosal implantation pocket 49 of FIG. 6. Upon placement of medical device 12 within sub-mucosal implantation pocket 49, push rod 46 is withdrawn from probe 22 via working channel 30. In some embodiments, a cauterization tool or suture tool can be introduced via working channel 30 to assist in closing the opening created in mucosal layer 16 by the implantation pocket 49. In other embodiments, the opening may be left to heal on its own. Medical device 12 remains in place, fixed within sub-mucosal layer 18. If medical device 12 is implanted within a working body lumen such as the esophagus, urethra or colon, the sub-mucosally implanted medical device may present very little obstruction to the body lumen.

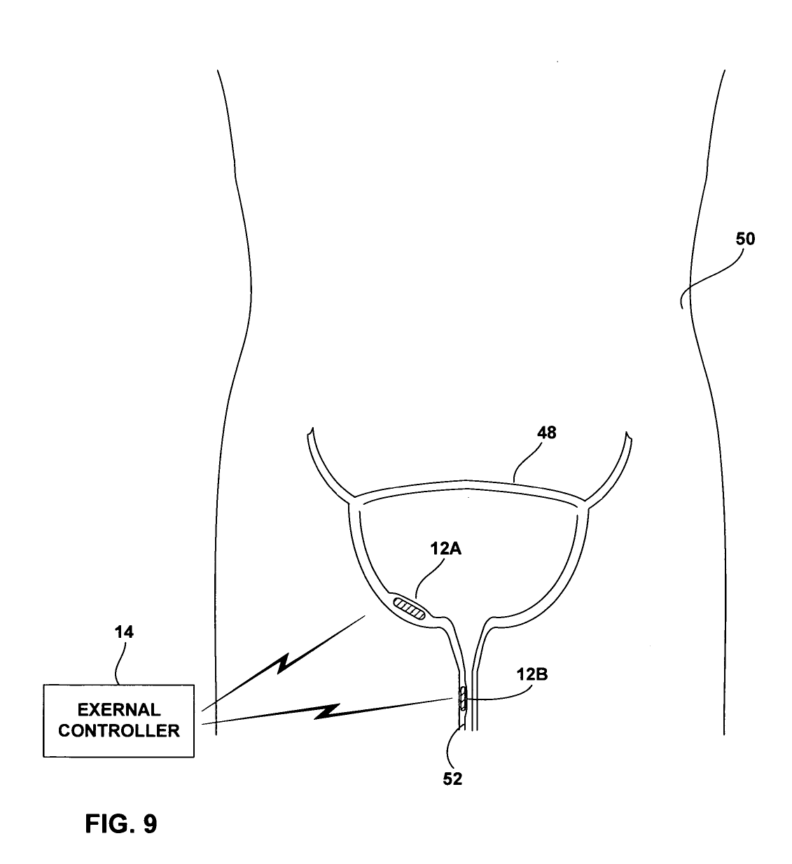

FIG. 9 is a diagram illustrating sub-mucosal implantation of medical devices 12A and 12B within the urinary tract of patient 50, i.e., within bladder 48 and urethra 52 of a patient 50. In the example of FIG. 9, an implantation instrument as described herein has been used to implant medical devices 12A and 12B within bladder 48 and urethra 52, respectively. Each medical device 12A, 12B includes a wireless telemetry interface to communicate with an external controller 14. Controller 14 may be an active controller that transmits commands or programs to control therapy delivered by one or both of medical devices 12A, 12B. Alternatively, controller 14 may primarily operate as a receiver for physiological information sensed and transmitted by medical devices 12A, 12B.

As an illustration, medical device 12A may be a contractile force sensor, such as a strain gauge, that sensed contractile force within the bladder muscle and transmits information based on the sensed force to external controller 14. As a further illustration, medical device 12B may be a Doppler flow sensor that ultrasonically detects flows within the urethra 52, and transmits sensed flow information to external controller 14. Alternatively, medical devices 12A, 12B may transmit information to other implanted devices, such as implanted stimulators positioned to deliver stimulation to the sacral nerves to alleviate urinary incontinence. In this manner, medical devices 12A, 12B may provide closed loop feedback relating to the state of the bladder 48 or urethra 52, which may be used as inputs for control of therapy delivered by the stimulators.

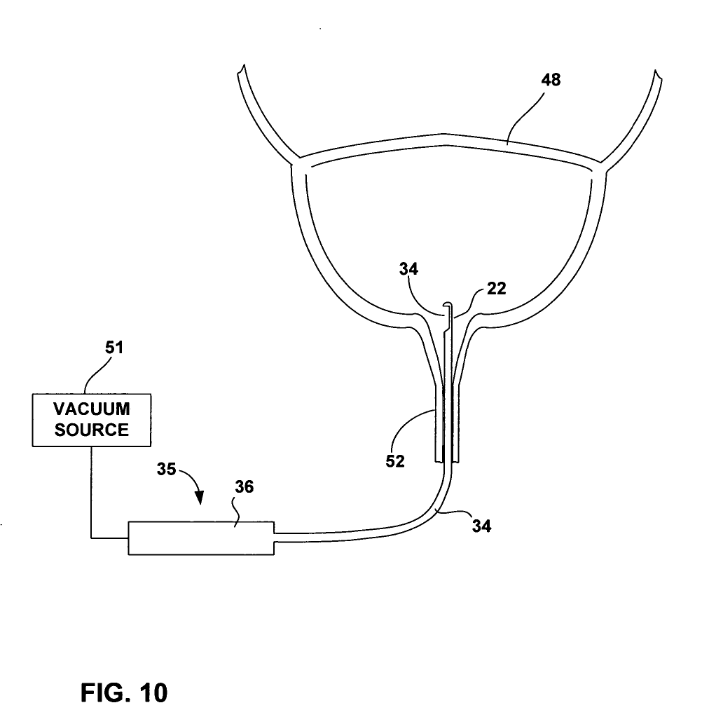

FIG. 10 is a diagram illustrating an implantation instrument 35 for implanting medical devices 12A, 12B within the bladder 48 and urethra 52, as shown in FIG. 9. Implantation instrument 35 may conform generally to the implantation instruments depicted in FIGS. 2-8. A vacuum source 51 is further shown in FIG. 10. Vacuum source 51 provides a source of vacuum pressure for the vacuum channel within elongated delivery tube 37. As shown in FIG. 10, probe 22 and elongated delivery tube 37 are introduced into urethra 52. Handle 36 and elongated delivery tube 37 may include a suitable steering mechanism to steer probe 22 toward a desired interior wall of bladder 48. Once probe is properly positioned, vacuum pressure is applied to draw mucosal tissue and sub-mucosal tissue into the vacuum cavity 34 of probe 22. Then, medical device 12 is implanted sub-mucosally as illustrated in FIGS. 6-8.

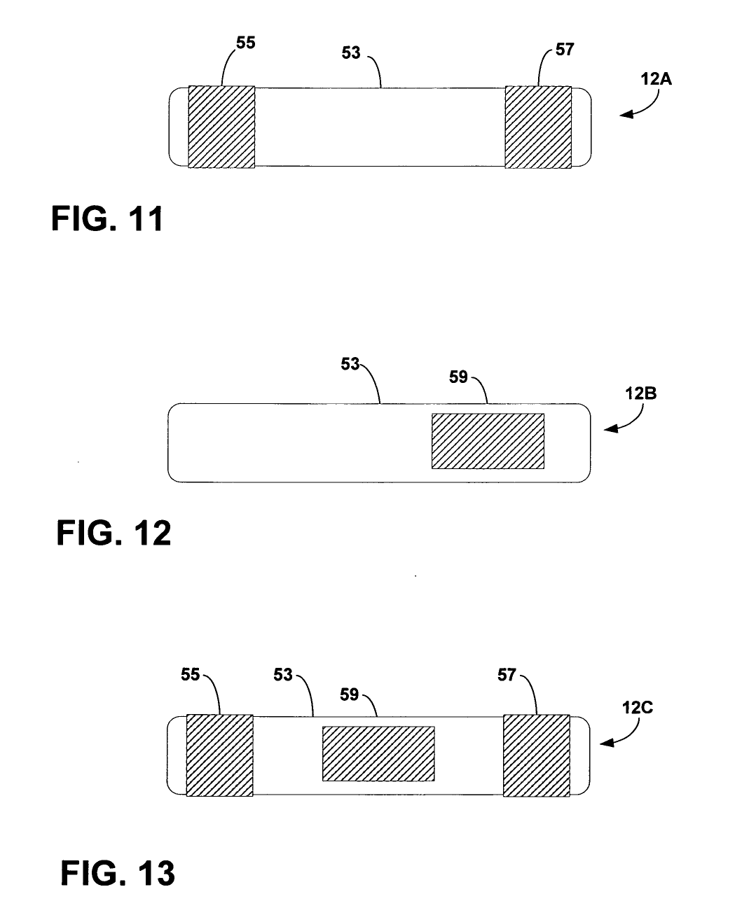

FIG. 11 is a schematic diagram illustrating an implantable stimulator 12A. As shown in FIG. 11, stimulator 12A is preferably a self-contained module, mounted within its own capsule-like housing 53. Housing 53 may be constructed from any of a variety of biocompatible materials, such as stainless steel or titanium. As will be described, housing 53 may carry one or more electrodes to permit delivery of electrical stimulation, an implantable pulse generator (IPG), and a telemetry interface to transmit or receive control signals or sensor signals. Although stimulator 14 may include short leads with electrodes that extend from the housing for placement proximate to a desired tissue or nerve site, the electrodes preferably are integrated with the stimulator.

In the example of FIG. 11, housing 53 carries a pair of electrodes 55, 57. Electrodes 55, 57 may be electrically conductive pads that are mounted on a particular surface of housing 53, or ring electrodes that extend about the entire periphery of the housing. Stimulator 12A includes an implantable pulse generator, and delivers neurostimulation therapy to patient 12 via electrodes 55, 57 in the form of electrical pulses generated by the implantable pulse generator. In some cases, housing 53 itself may form an active “can” electrode.

In alternative embodiments, stimulator 12A may include a single electrode for coordinated operation with an electrode on another stimulator. In this case, two stimulators 12A may function to form a bipolar stimulation arrangement. Alternatively, a stimulator 12A may include two or more electrodes that form a bipolar or multi-polar stimulation arrangement. Hence, stimulators 12A may deliver neurostimulation energy independently of other stimulators or in a coordinated manner with other stimulators. In either case, the electrode or electrodes 55, 57 may be formed on the housing 53 of stimulator 12A.

To facilitate implantation within sub-mucosal layer 18, housing 53 has relatively small dimensions. In some embodiments, the capsule-like housing 53 may be substantially cylindrical, with a length greater than its diameter and flat or rounded ends, although the invention is not limited to any particular shape. If constructed as a cylindrical capsule, housing 53 may have a length of less than approximately 25 mm, and preferably approximately 10 to 25 mm, and a diameter of less than or equal to approximately 10 mm, and preferably approximately 3 to 10 mm. Again, other shapes for housing 53 are possible. In general, it is desirable that housing 53 present a profile or depth, taken in a direction perpendicular to mucosal layer 16, of less than or equal to approximately 10 mm, and more preferably approxiamtely 3 to 10 mm, to facilitate implantation between mucosal layer 16 and muscle layer 20. These dimensions for housing 53 may apply to the housings depicted in FIGS. 12 and 13 as well.

FIG. 12 is a schematic diagram illustrating an implantable sensing device 12B. Like stimulator 12A, sensing device 12B preferably is a self-contained module having a housing 53. Again, housing 53 may be constructed from a biocompatible metal, such as titanium. A sensor 59 is mounted on or exposed by housing 53 to sense physiological conditions within patient 12 in the vicinity of the sensor. Sensor 59 is configured to sense physiological conditions within the sub-mucosal layer 18, or within a body lumen or organ via mucosal layer 16. For example, sensor 59 may sense electrical potentials, such as electromyographic potentials, to sense a patient response to nerve stimulation, or sense the onset of an event such as urinary voiding. As another example, sensor 59 may operate as an ultrasonic Doppler flow sensor to detect flow within a body lumen, such as the urethra, by transmitting and receiving ultrasonic waves via mucosal layer 16.

For thin mucosal layers 16, optical sensors also may be useful, e.g., to sense flow or presence of fluid within a body lumen on a side of the mucosal layer opposite sensor 59. In this case, light may be transmitted through mucosal layer 16 and reflected from fluid within the body lumen. Alternatively, sensing devices 12B may be implanted on opposite sides of a body lumen for transmissive sensing of light through the fluid medium. In this case, one sensing device 12B operates as an emitter, while the other acts as a receiver. Temperature sensors also may be useful to detect the presence or flow of fluid, such as urine or blood, within a body lumen on a side of mucosal layer 16 opposite the sensor.

FIG. 13 is a schematic diagram illustrating an implantable module 12C incorporating both a stimulator and a sensor. In the example of FIG. 5, a housing 53 carries both electrodes 55, 57 and a sensor 59 to provide stimulation and sensing functionality within a single module. Integration of stimulation and sensing in a single module may be desirable in some applications. In other applications, however, it will be advantageous to sense physiological conditions at a location remote from the site of stimulation, in order to assess the response of a patient to the stimulation. Other types of sensors may be used, such as sensors capable of sensing pressure, contractile force, flow, electromyographic potentials, temperature, pH, the presence of particular chemicals, and the like. Such sensors may be deployed at a variety of locations within a patient. For applications directed to therapy for sexual dysfunction, sensor 59 may be configured to sense other physiological parameters such as penile blood pressure, penile blood flow, penile size, tumescence, or the like.

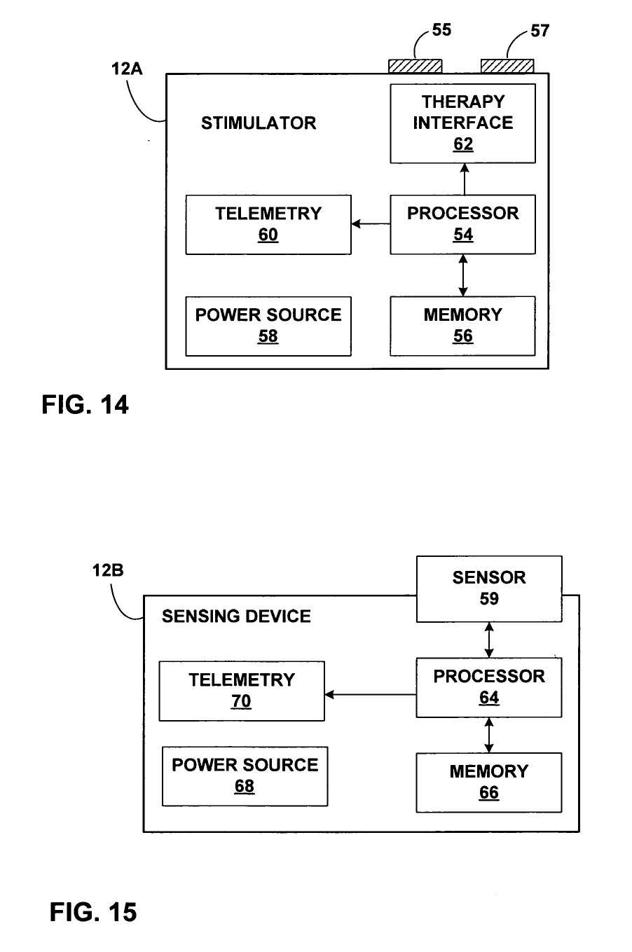

FIG. 14 is a block diagram illustrating various components of an implantable stimulator 12A for sub-mucosal implantation within a patient. In the example of FIG. 14, a stimulator 12A includes a housing carrying a pair of electrodes 55, 57, which can be referenced to each other to form a bipolar arrangement. Stimulator 12A further may include a processor 54, memory 56, power source 58, telemetry interface 60, and therapy delivery interface 62. Electrodes 55, 57 are electrically coupled to a therapy delivery interface 62, which includes an implantable pulse generator to generate stimulation pulses. Power source 58 may be a battery, which may be non-rechargeable. Alternatively, the battery may be rechargeable with power delivered from an external charging device via an inductive power interface. As a further alternative, stimulator 12A may be inductively powered by an external device.

Processor 54 controls the implantable pulse generator within therapy delivery interface 62 to deliver stimulation according to selected stimulation parameters. Specifically, processor 54 controls therapy delivery interface 62 to deliver electrical pulses with selected voltage or current amplitudes, pulse widths, frequencies, and durations specified by programs stored in memory 56. In addition, processor 54 may control therapy delivery interface 62 to deliver neurostimulation pulses via one or both of electrodes 55, 57 with selected polarities. In some embodiments, two or more electrodes may be provided on the housing of stimulator 12A.

In addition, processor 54 may control therapy delivery interface 62 to deliver each pulse according to a different program, thereby interleaving programs to simultaneously treat different symptoms or provide a combined therapeutic effect. For example, in addition to treatment of one type of disorder, such as sexual dysfunction, stimulator 12A may be configured to deliver neurostimulation therapy to simultaneously treat pain or incontinence. Processor 54 may include a microprocessor, a controller, a digital signal processor (DSP), an application specific integrated circuit (ASIC), a field programmable gate array (FPGA), or other equivalent logic circuitry, or the like.

In some embodiments, memory 56 stores multiple sets of stimulation parameters that are available to be selected by the patient for delivery of neurostimulation therapy. For example, memory 56 may store stimulation parameters transmitted by an external clinician programmer. As described herein, the stimulation parameters may be formulated for treatment during distinct phases of sexual activity, such as a first phase involving arousal and a second phase involving orgasm. An external programmer may communicate with stimulator 12A by wireless telemetry to permit a patient to adjust neurostimulation delivered by the stimulator, and select different neurostimulation parameters.

Memory 56 also stores program instructions that, when executed by processor 32, cause stimulator 12A to deliver neurostimulation therapy. Memory 56 may include any volatile or non-volatile media, such as a RAM, ROM, NVRAM, EEPROM, flash memory, and the like, or any combination thereof. Accordingly, the invention also contemplates computer-readable media storing instructions to cause processor 54 to provide the functionality described herein. In some embodiments, to further reduce the size of stimulator 12A, the stimulator may include integrated or discrete logic circuitry instead of memory 56 and a programmable processor 54.

Telemetry interface 60 supports wireless communication between stimulator 12A and an external clinician programmer or patient programmer for programming of the stimulator. Also, telemetry interface 60 may support communication with other stimulators or sensors implanted within the patient. Accordingly, telemetry interface 60 includes appropriate amplification, filtering, and modulation circuitry, as well as an antenna such as an inductive coil antenna. A handheld computing device (not shown) may be provided as a programmer to permit a clinician to program neurostimulation therapy into stimulators 12A for the patient, e.g., using input keys and a display. Using the external programmer, the clinician may specify neurostimulation parameters for use in the different phases of physiological activity by the patient. The external programmer supports radio frequency telemetry with stimulator 12A to download neurostimulation parameters and, optionally, upload operational or physiological data from stimulator 12A or implanted sensors. In this manner, a clinician may periodically interrogate an implanted system of stimulators and sensors to evaluate efficacy and, if necessary, modify stimulation parameters.

In some embodiments, a master controller may be integrated with an external patient programmer or a stimulator 12A. Like the clinician programmer, a patient programmer can be provided as a handheld computing device. The patient programmer may include a display and input keys to allow the patient to interact with the patient programmer. In this manner, the patient programmer provides the patient with an interface for control of neurostimulation therapy by stimulator 12A. For example, the patient may use the patient programmer to start, stop or adjust neurostimulation therapy. In particular, the patient programmer may permit the patient to adjust stimulation parameters such as amplitude, frequency, pulse width and duration, within an adjustment range specified by the clinician via a clinician programmer.

Telemetry interface 60 also may support wireless communication with one or more wireless sensors 12B that sense physiological signals and transmit the signals to stimulator 12A. Hence, stimulator 12A may be directly responsive to physiological signals generated by sensing devices 12B. Alternatively, a master controller may receive the physiological signals and transmit control signals to stimulator 12A. For example, in response to detection of a particular physiological condition or level, the master controller may adjust the neurostimulation therapy delivered by stimulator 12A and other implanted stimulators, as applicable. Again, the master controller may reside within an external controller or be integrated with a stimulator.

FIG. 15 is a block diagram illustrating various components of a sensing device 12B for sub-mucosal implantation within a patient. As shown in FIG. 7, sensing device 12B may include a processor 64, memory 66, power source 68, telemetry interface 70 and physiological sensor 59. Processor 64 and memory 66 may not be necessary in some embodiments. Instead, sensing device 12B may simply provide a sensor 59 and telemetry interface 70 equipped to transmit a raw, unprocessed sensor signal to stimulator 12A or a controller. In general, processor 64, memory 66, power source 68, and telemetry interface 70 may be constructed like similar components within stimulator 12B, as described above with reference to FIG. 14. Sensor 59 may be selected for any of a variety of sensing applications, and may include appropriate signal processing circuitry such as amplifier, filter, and analog-to-digital conversion circuitry for presentation of sensed information to processor 64.

Again, in various embodiments, sensing device 12B may take a variety of forms sufficient to sense desired physiological conditions including pressure sensors, flow sensors, temperature sensors, electromyographic sensors, pH sensors, chemical sensors. Hence, in terms of structure, sensor 59 may include strain gauge sensors, optical sensors, ultrasonic sensors, piezoelectric sensors, electrical sensors, reactive sensors, protein sensors, or the like. As an illustration, for urinary incontinence applications, one or more sensing devices 12B may be implanted to sense bladder pressure, bladder contractile force, urine level, urethral pressure, urethral flow, urine presence within the urethra or other parameters indicating the state of bladder function. In this manner, a sensing device 12B can provide an indication of the onset, state or progress of a voiding event, or a response of the patient to neurostimulation therapy, to trigger or adjust neurostimulation therapy.

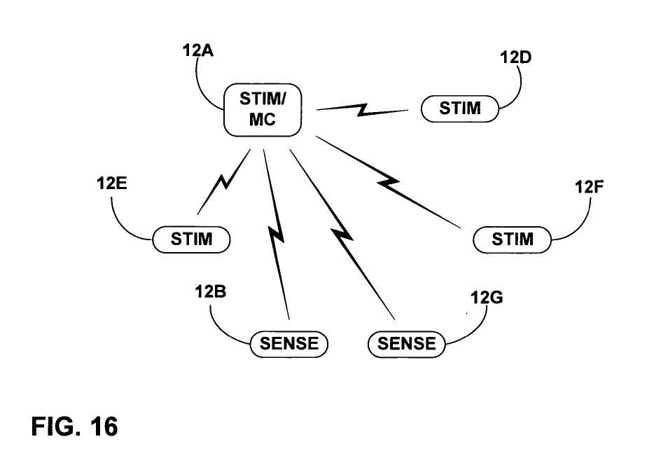

FIG. 16 is a block diagram illustrating an array of sub-mucosally implanted stimulators and sensors. More particularly, FIG. 16 illustrates a master-slave arrangement of implanted stimulators and sensors. A master controller (MC) is integrated with a stimulator to form an integrated stimulator/master controller (STIM/MC) 12A. Alternatively, the master controller may be a separately implanted device, an external device carried by the patient, or a controller integrated with an implanted sensing device. Integrated stimulator/master controller 12A acts as both a stimulator and a “master” controller for other “slave” stimulators 12D, 12E, 12F. Stimulator 12A, as master controller, may be responsive to information received from implanted sensing devices 12B, 12G to generate control signals. Also, stimulator 12A may activate sensing 12B, 12G to obtain physiological information.

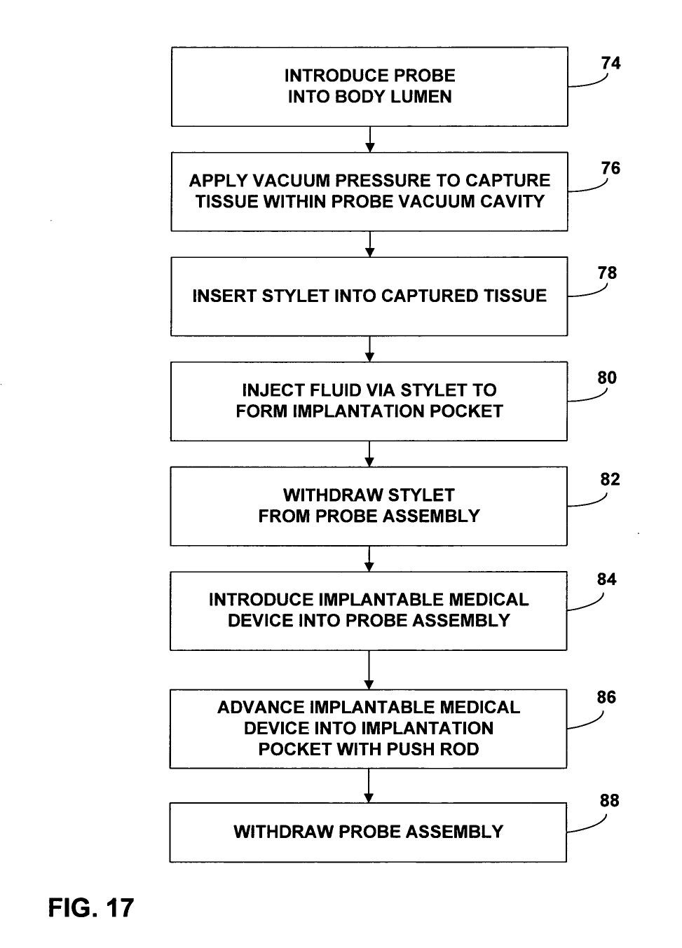

FIG. 17 is a flow diagram illustrating a technique for sub-mucosal implantation of medical devices. As shown in FIG. 17, probe 22 is introduced into a body lumen (74), which may be a natural body lumen, or an access lumen created by surgical or laparoscopic techniques. Upon application of vacuum pressure to capture mucosal and sub-mucosal tissue within vacuum cavity 34 (76), a hollow stylet is inserted into the captured tissue (78) via working channel 30. Fluid such as saline is then injected into sub-mucosal layer 18 to form an implantation pocket (80).

Upon withdrawal of the stylet from the probe assembly (82), an implantable medical device, such as a stimulator or sensor, is introduced into the probe assembly via the working channel 30 (84). The implantable medical device is advanced into the implantation pocket with a push rod via the working channel 30 (86), and the probe assembly is withdrawn (88). In some applications, a cauterization or suture tool optionally may be introduced to close the opening to the implantation pocket. Once the implantable medical device is implanted, the implantation process may continue if additional implants are desired. For example, the probe assembly may be repositioned without withdrawal from the body lumen. In this case, another implantable medical device may be introduced at another implant site.

The preceding specific embodiments are illustrative of the practice of the invention. It is to be understood, therefore, that other expedients known to those skilled in the art or disclosed herein may be employed without departing from the invention or the scope of the claims. For example, the present invention further includes within its scope methods of making and using systems as described herein. Also, the invention may facilitate implantation of not only implantable stimulators or sensors, but also other devices such as drug delivery devices.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Various embodiments of the invention have been described. These and other embodiments are within the scope of the following claims.

1. A method for implanting a medical device, the method comprising:
applying vacuum pressure to a mucosal layer to draw a portion of the mucosal tissue layer and a portion of a sub-mucosal layer into a vacuum cavity defined by an implantation instrument; and
introducing a medical device into the portion of the sub-mucosal tissue layer drawn into the vacuum cavity, wherein the medical device includes an electrically powered medical device.

2. The method of claim 1, wherein applying vacuum pressure comprises applying the vacuum pressure directly to the mucosal layer via a vacuum port associated with the implantation instrument.

3. The method of claim 1, wherein introducing a medical device includes injecting fluid into the portion of the sub-mucosal tissue layer to form an implantation pocket, and placing the medical device within the implantation pocket.

4. The method of claim 3, wherein injecting fluid includes advancing a hollow stylet along a length of a working channel within the implantation instrument, forcing a distal tip of the stylet into the portion of the sub-mucosal tissue layer, and injecting the fluid via the stylet.

5. The method of claim 3, wherein introducing the medical device further includes withdrawing the stylet, advancing a push rod along the length of the working channel within the implantation instrument to push the medical device into the implantation pocket.

6. The method of claim 1, wherein the medical device includes an electrical stimulator.

7. The method of claim 6, wherein the electrical stimulator includes one or more electrodes, a pulse generator, a power source, and a wireless telemetry interface.

8. The method of claim 6, wherein the stimulator is a neurostimulator.

9. The method of claim 1, wherein the medical device includes a substantially capsule-shaped housing sized for implantation within the sub-mucosal tissue layer.

10. The method of claim 1, wherein the medical device includes a physiological sensing device.

11. The method of claim 10, wherein the sensing device includes a sensor, a power source, and a wireless telemetry interface.

12. The method of claim 1, wherein the vacuum cavity has a depth of approximately 5 to approximately 15 mm.

13. The method of claim 12, wherein the vacuum cavity has a length of approximately 5 to approximately 40 mm and a width of approximately 5 to approximately 15 mm.

14. The method of claim 1, wherein the implantation instrument defines a working channel that defines an opening in the vacuum cavity, and the opening is disposed at a depth selected for access to the sub-mucosal tissue layer.

15. The method of claim 1, further comprising repetitively applying vacuum pressure to additional portions of the mucosal tissue layer and introducing additional medical devices to implant a plurality of medical devices in different portions of the sub-mucosal tissue layer.

16. The method of claim 15, wherein at least one of the medical devices includes an electrical stimulator and at least one of the medical devices includes a physiological sensing device.

17. A system for implantation of a medical device, the system comprising:
an elongated delivery tube;
a probe coupled to a distal end of the delivery tube;
a vacuum cavity defined by the probe to apply vacuum pressure to a mucosal layer to draw a portion of the mucosal tissue layer and a portion of a sub-mucosal layer into a vacuum cavity defined by an implantation instrument;
a vacuum channel to apply the vacuum pressure to the vacuum cavity;
an electrically powered medical device; and
a working channel to introduce the medical device into the portion of the sub-mucosal tissue layer drawn into the vacuum cavity.

18. The system of claim 17, further comprising a plurality of vacuum ports defined by the vacuum cavity to apply the vacuum pressure directly to the mucosal tissue layer.

19. The system of claim 17, further comprising a hollow stylet, for introduction along a length of the working channel, to inject fluid into the portion of the sub-mucosal tissue layer to form an implantation pocket.

20. The system of claim 19, further comprising a push rod, for introduction along the length of the working channel, to push the medical device into the implantation pocket.

21. The system of claim 17, wherein the medical device includes an electrical stimulator.

22. The system of claim 21 wherein the electrical stimulator includes one or more electrodes, a pulse generator, a power source, and a wireless telemetry interface.

23. The system of claim 21, wherein the stimulator is a neurostimulator.

24. The system of claim 17, wherein the stimulator includes a substantially capsule-shaped housing sized for implantation within the sub-mucosal tissue layer.

25. The system of claim 17, wherein the medical device includes a physiological sensing device.

26. The system of claim 25, wherein the sensing device includes a sensor, a power source, and a wireless telemetry interface.

27. The system of claim 17, wherein the vacuum cavity has a depth of approximately 5 to approximately 15 mm.

28. The system of claim 27, wherein the vacuum cavity has a length of approximately 5 to approximately 40 mm and a width of approximately 5 to approximately 15 mm.

29. The system of claim 17, wherein the working channel defines an opening in the vacuum cavity, and the opening is disposed at a depth selected for access to the sub-mucosal tissue layer.

30. A system for implantation of a medical device, the system comprising:
an electrically powered implantable medical device; and
an implantation instrument including:
tissue securing means,
means for drawing mucosal tissue and sub-mucosal tissue into the tissue securing means, and
means for introducing the medical device into the sub-mucosal tissue drawn into the tissue securing means.

31. The system of claim 30, wherein the drawing means includes means for applying vacuum pressure to the mucosal tissue.

32. The system of claim 30, wherein the introducing means includes a working channel defined by the implantation instrument and a push rod to push the medical device through the working channel and into the sub-mucosal tissue drawn into the tissue securing means.

33. The system of claim 30, wherein the tissue securing means includes a vacuum cavity, and the means for drawing mucosal tissue and sub-mucosal tissue into the tissue securing means includes means for applying vacuum pressure to the vacuum cavity.

34. The system of claim 30, wherein the introducing means further includes a stylet means for injecting fluid into the portion of the sub-mucosal tissue layer to form an implantation pocket.

35. The system of claim 30, wherein the medical device includes an electrical stimulator.

36. The system of claim 35 wherein the electrical stimulator includes one or more electrodes, a pulse generator, a power source, and a wireless telemetry interface.

37. The system of claim 35, wherein the stimulator is a neurostimulator.

38. The system of claim 30, wherein the medical device includes a substantially capsule-shaped housing sized for implantation within the sub-mucosal tissue layer.

39. The system of claim 30, wherein the medical device includes a physiological sensing device.

40. The system of claim 30, wherein the sensing device includes a sensor, a power source, and a wireless telemetry interface.
